(19) United States
(12) Patent Application Publication
(10) Pub. No.: US 2023/0414883 A1
(43) Pub. Date: Dec. 28, 2023

(54) NEBULIZER

(71) Applicant: Murata Manufacturing Co., Ltd., Kyoto (JP)

(72) Inventors: Miki IKEDA, Kyoto (JP); Nobuhira TANAKA, Kyoto (JP); Hiroaki WADA, Kyoto (JP)

(21) Appl. No.: 18/462,601

(22) Filed: Sep. 7, 2023

Related U.S. Application Data
(63) Continuation of application No. PCT/JP2022/013663, filed on Mar. 23, 2022.

(30) Foreign Application Priority Data
Mar. 25, 2021 (JP) 2021-051079

(51) Int. Cl. A61M 11/04

(52) U.S. Cl. CPC A61M 11/042

(57) ABSTRACT

A nebulizer includes a housing and a mouthpiece. The housing surrounds a pump for spraying a liquid reagent out of the nebulizer. An opening is formed in the housing. The inside and the outside of the housing communicate with each other through the opening. Arrow indicates an air flow from the opening to an inlet of the pump. The air flow indicated by arrow passes by the outer surface of the pump

### CROSS REFERENCE TO RELATED APPLICATION

This is a continuation of International Application No. PCT/JP2022/013663 filed on Mar. 23, 2022 which claims priority from Japanese Patent Application No. 2021-051079 filed on Mar. 25, 2021. The contents of these applications are incorporated herein by reference in their entireties.

### BACKGROUND OF THE DISCLOSURE

### Field of the Disclosure

The present disclosure relates to nebulizers, and in particular, to nebulizers utilizing compressed air.

### Description of the Related Art

Various techniques have been proposed in relation to nebulizers utilizing compressed air. For example, Japanese Unexamined Patent Application Publication No. 2015-159991 (Patent Document 1) discloses a nebulizer including an atomizing section for generating aerosol and a technique of selecting and adjusting liquid particles contained in the aerosol.
Patent Document 1: Japanese Unexamined Patent Application Publication No. 2015-159991

### BRIEF SUMMARY OF THE DISCLOSURE

In a type of nebulizer accommodating a pump to generate compressed air, the pump may generate heat when actuated, which may lead to malfunctions, such as a failure of the pump.

A possible benefit of the present disclosure is to provide a technique for dissipating the heat of a heat generating element, such as a pump or a circuit board, in a nebulizer.

According to an embodiment of the present disclosure, a nebulizer includes a pump for spraying a liquid reagent and a housing that surrounds the pump. The housing has a space defined at least partially by an outer surface of the pump and an element inside the housing. At least one opening is provided to enable the space to communicate with the outside of the housing.

According to the present disclosure, the heat emission from the heat generating element, such as the pump or the circuit board, can be promoted.

### BRIEF DESCRIPTION OF THE SEVERAL VIEWS OF THE DRAWINGS

FIG. 1 is a schematic view illustrating a structure of a nebulizer according to a first embodiment.

FIG. 2 is a schematic view illustrating a structure of a nebulizer according to a second embodiment.

FIG. 3 is a schematic view illustrating a structure of a nebulizer according to a third embodiment.

FIG. 4 is a schematic view illustrating a structure of a nebulizer according to a fourth embodiment.

FIG. 5 is a schematic view illustrating a structure of a nebulizer according to a fifth embodiment.

FIG. 6 is a schematic view illustrating a structure of a nebulizer according to a sixth embodiment.

FIG. 7 is a schematic view illustrating a structure of a nebulizer according to a seventh embodiment.

FIG. 8 is a schematic view illustrating a structure of a nebulizer according to an eighth embodiment.

FIG. 9 is a schematic view illustrating a structure of a nebulizer according to a ninth embodiment.

FIG. 10 is a schematic view illustrating a structure of a nebulizer according to a tenth embodiment.

FIG. 11 is a schematic view illustrating a structure of a nebulizer according to an eleventh embodiment.

FIG. 12 is a schematic view illustrating a structure of a nebulizer according to a twelfth embodiment.

FIG. 13 is a schematic view illustrating a structure of a nebulizer according to a thirteenth embodiment.

FIG. 14 is a view illustrating the nebulizer of the thirteenth embodiment in a state in which a valve 53 is rotated from a state illustrated in FIG. 13.

### DETAILED DESCRIPTION OF THE DISCLOSURE

Nebulizers according to some embodiments will be described in detail with reference to the drawings. Note that the same reference signs denote the same or similar elements.

### First Embodiment

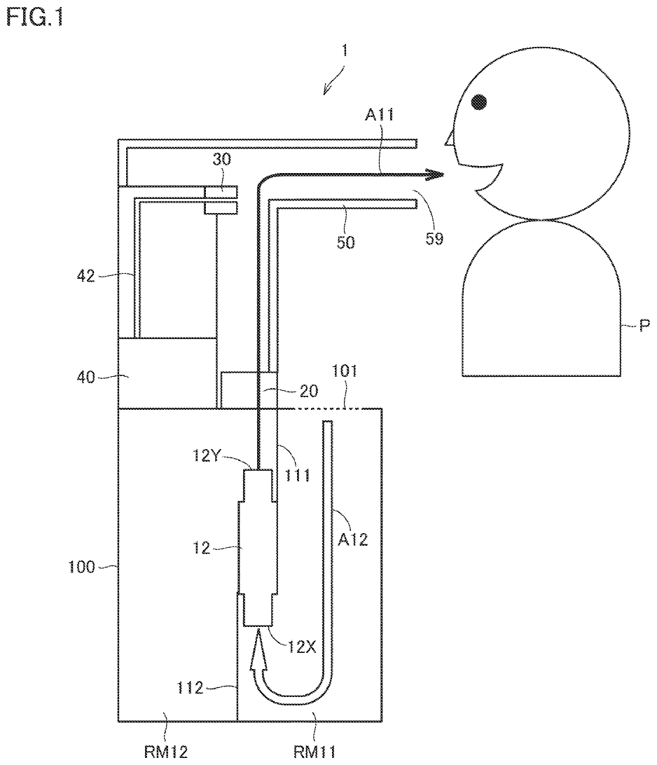

FIG. 1 is a schematic view illustrating a structure of a nebulizer of a first embodiment according to the present disclosure. As illustrated in FIG. 1, a nebulizer 1 includes a housing 100 that covers a main body of the nebulizer 1 and also includes a mouthpiece 50 that serves as a classification cover. The mouthpiece 50 has an outlet 59, which is an example of an outlet through which an aerosol is discharged. Note that the aerosol discharged by the nebulizer 1 is not limited to that taken orally. The classification cover, which has the outlet 59 through which the aerosol is discharged, can be formed as a nosepiece or a mask.

The housing 100 accommodates a pump 12. The pump 12 has an air inlet 12X and an air outlet 12Y. The pump 12 supplies compressed air for atomizing and discharging a liquid medicine. The housing 100 may also accommodate a battery for supplying electric power to drive the pump 12, a control circuit for controlling the pump 12, and/or other elements.

The nebulizer 1 includes a liquid medicine tank 40, an atomizing nozzle 30, an air nozzle 20, and piping 42, which are attached to the main body of the nebulizer 1. The compressed air produced by the pump 12 is supplied through the air nozzle 20 to the mouthpiece 50. A liquid medicine contained in the liquid medicine tank 40 is supplied to the atomizing nozzle 30 through the piping 42 and is atomized at the atomizing nozzle 30. More specifically, the liquid medicine is sucked up to the tip end of the atomizing nozzle 30 and atomized by an air flow from the air nozzle 20 and is further discharged through the outlet 59 of the mouthpiece 50 to a patient P. The nebulizer 1 also includes a structure (not illustrated) to liquefy the mist not discharged out of the outlet 59 and returns the mist into the liquid medicine tank 40.

In the pump 12, the inlet 12X faces downward, and the outlet 12Y faces upward. Arrow A11 indicates a fluid flow from the outlet 12Y to the outlet 59 of the mouthpiece 50.

Partition plates 111 and 112 are formed inside the housing 100. The partition plates 111 and 112 and the pump 12 separate the inside of the housing 100 into a space RM11 and a space RM12.

An opening 101 is provided at a top surface of the housing 100. The opening 101 corresponds to a first opening. The space RM11 communicates with the outside of the housing 100 through the opening 101. Arrow A12 indicates an air flow from the opening 101 to the inlet 12X through the space RM11. As indicated by arrow A12, the air introduced through the opening 101 into the space RM11 passes by the outer surface of the pump 12 toward the inlet 12X. At least the air flow indicated by arrow A12 can promote the heat emission from the pump 12.

### Second Embodiment

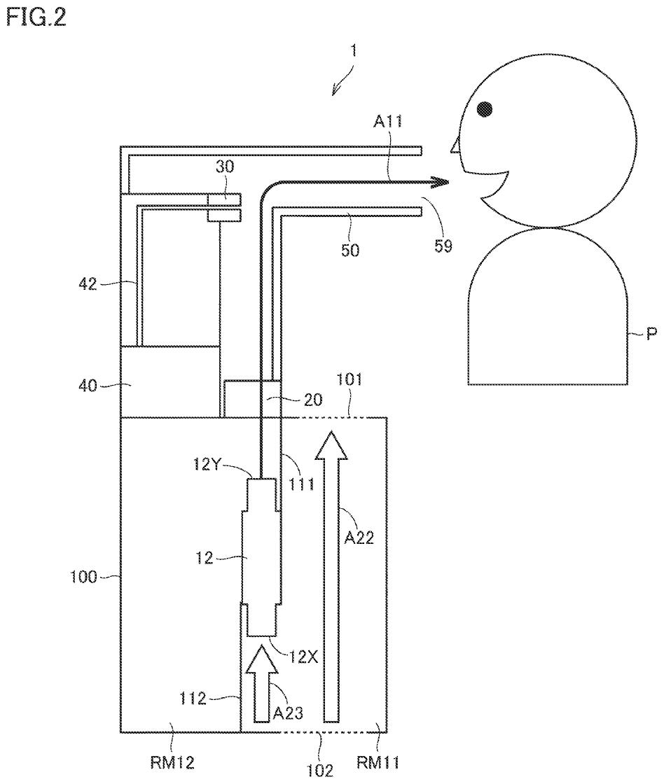

FIG. 2 is a schematic view illustrating a structure of a nebulizer of a second embodiment according to the present disclosure. In the example illustrated in FIG. 2, the housing 100 also has an opening 102 formed at the bottom surface thereof. The opening 102 corresponds to a second opening.

Arrow A22 indicates an air flow from the opening 102 to the opening 101 passing by the outer surface of the pump 12. At least the air flow indicated by arrow A22 can promote the heat emission from the pump 12. Arrow A23 indicates an air flow from the opening 102 to the inlet 12X. At least the air flow indicated by arrow A23 can also promote the heat emission from the pump 12. In the case of the housing 100 having the openings formed at respective surfaces being opposite to each other, as shown as the openings 101 and 102, an air flow is expected to occur from one of the openings toward the other as indicated by arrow A22. Even if multiple openings are formed at the same surface of the housing 100, the amount of the air to be introduced into the housing 100 is at least expected to increase.

### Third Embodiment

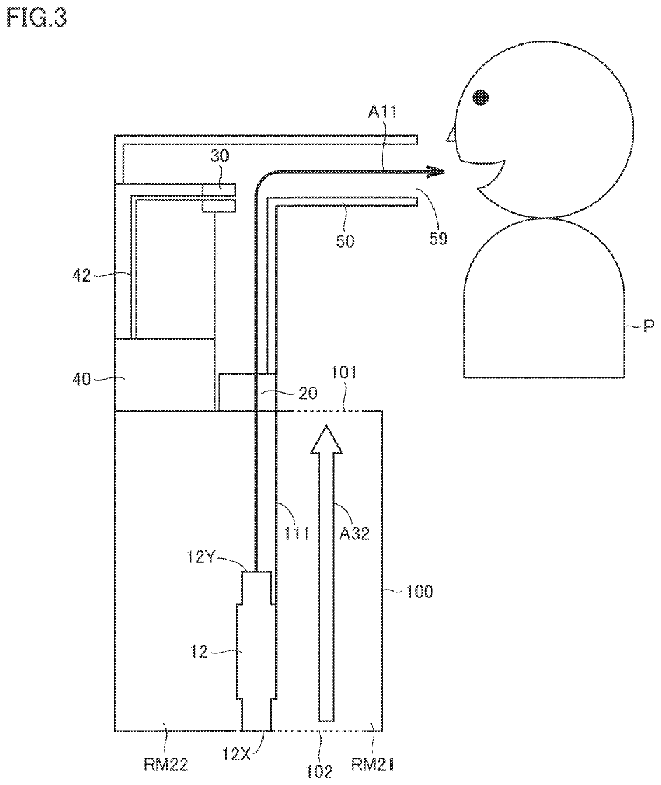

FIG. 3 is a schematic view illustrating a structure of a nebulizer of a third embodiment according to the present disclosure. In the example illustrated in FIG. 3, the opening 102 is made wider than that in FIG. 2. In the example illustrated in FIG. 2, the pump 12 is disposed such that the inlet 12X is exposed to the space RM11. On the other hand, in the example illustrated in FIG. 3, the pump 12 is disposed such that the inlet 12X is exposed to the opening 102. Accordingly, the air outside the housing 100 is directly introduced to the pump 12 through the inlet 12X.

In FIG. 3, the inside of the housing 100 is separated into a space RM21 and a space RM22 by the partition plate 111 and the pump 12. Arrow A32 indicates an air flow from the opening 102 to the opening 101 passing by the outer surface of the pump 12. At least the air flow indicated by arrow A32 can promote the heat emission from the pump 12.

### Fourth Embodiment

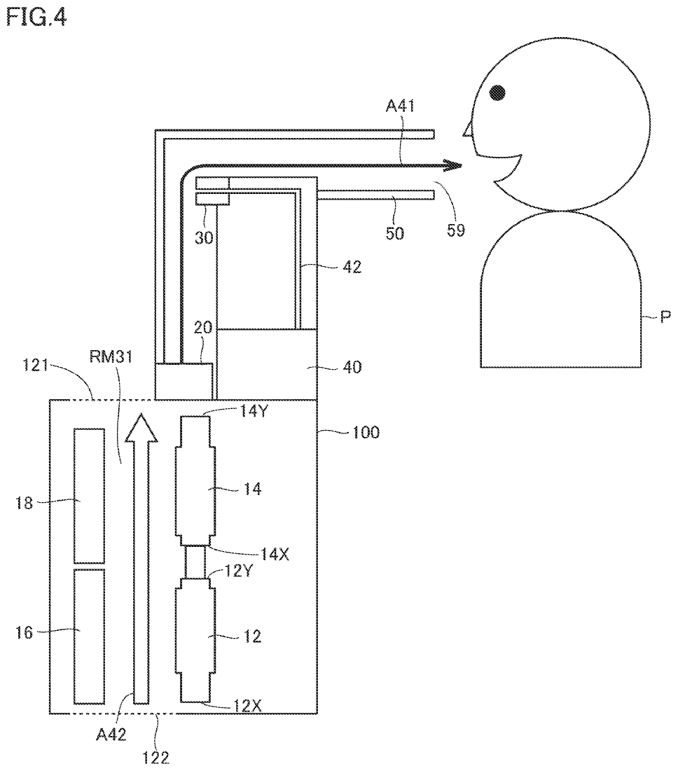

FIG. 4 is a schematic view illustrating a structure of a nebulizer of a fourth embodiment according to the present disclosure. In the example illustrated in FIG. 4, the nebulizer 1 further includes a pump 14 in addition to the pump 12. The pump 14 has an inlet 14X and an outlet 14Y. A pipe connects the outlet 12Y of the pump 12 to the inlet 14X of the pump 14. The air compressed by the pump 12 and by the pump 14 is supplied to the air nozzle 20. Arrow A41 indicates a fluid flow from the air nozzle 20 to the outlet 59 of the mouthpiece 50.

In the example illustrated in FIG. 4, a board 16 and a board 18 are disposed inside the housing 100. The board 16 includes a control circuit for controlling the pump 12, and the board 18 includes a control circuit for controlling the pump 14. An opening 121 is formed at the top surface of the housing 100, and an opening 122 is formed at the bottom surface of the housing 100.

A space RM31 is defined at least partially by the outer surfaces of the pumps 12 and 14 and the boards 16 and 18, and the space RM31 communicates with the outside of the housing 100 through the openings 121 and 122. Arrow A42 indicates an air flow from the opening 122 to the opening 121 passing by the outer surfaces of the pumps 12 and 14 and the boards 16 and 18. At least the air flow indicated by arrow A42 can promote the heat emission from the pump 12. Note that one control board may control multiple pumps in the nebulizer 1. In this case, the openings 121 and 122 can be formed so as to enable an air flow to occur from the opening 122 to the opening 121 passing by the outer surfaces of the one control board and the multiple pumps.

### Fifth Embodiment

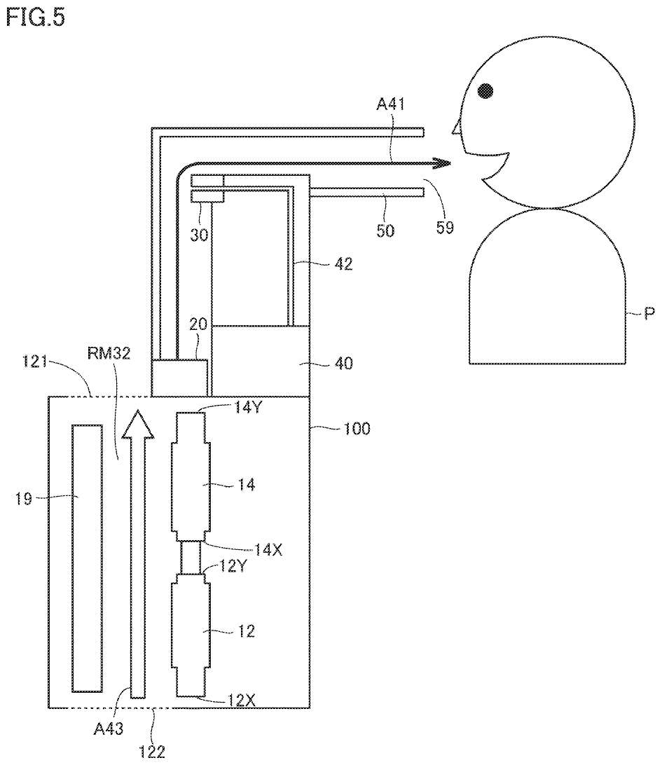

FIG. 5 is a schematic view illustrating a structure of a nebulizer of a fifth embodiment according to the present disclosure. In the example illustrated in FIG. 5, a battery 19 is also disposed in the housing 100. The battery 19 supplies electric power to drive the pump 12 and the pump 14. The opening 121 is formed at the top surface of the housing 100, and the opening 122 is formed at the bottom surface of the housing 100.

A space RM32 is defined at least partially by the outer surfaces of the pumps 12 and 14 and the battery 19, and the space RM32 communicates with the outside of the housing 100 through the openings 121 and 122. Arrow A43 indicates an air flow from the opening 122 to the opening 121 passing by the outer surfaces of the pumps 12 and 14 and the battery 19. At least the air flow indicated by arrow A43 can promote the heat emission from the pump 12.

### Sixth Embodiment

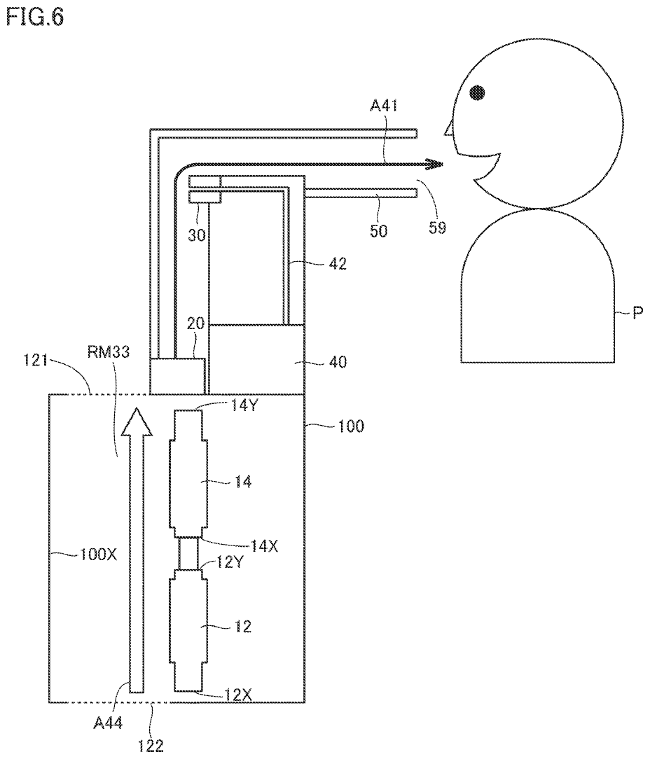

FIG. 6 is a schematic view illustrating a structure of a nebulizer of a sixth embodiment according to the present disclosure.

In the example illustrated in FIG. 6, a space RM33 is defined at least partially by the outer surfaces of the pumps 12 and 14 and an inner surface (inside surface) 100X of the housing 100. The space RM33 communicates with the outside of the housing 100 through the openings 121 and 122. Arrow A44 indicates an air flow from the opening 122 to the opening 121 passing by the outer surfaces of the pumps 12 and 14 and the inner surface 100X. At least the air flow indicated by arrow A44 can promote the heat emission from the pump 12.

### Seventh Embodiment

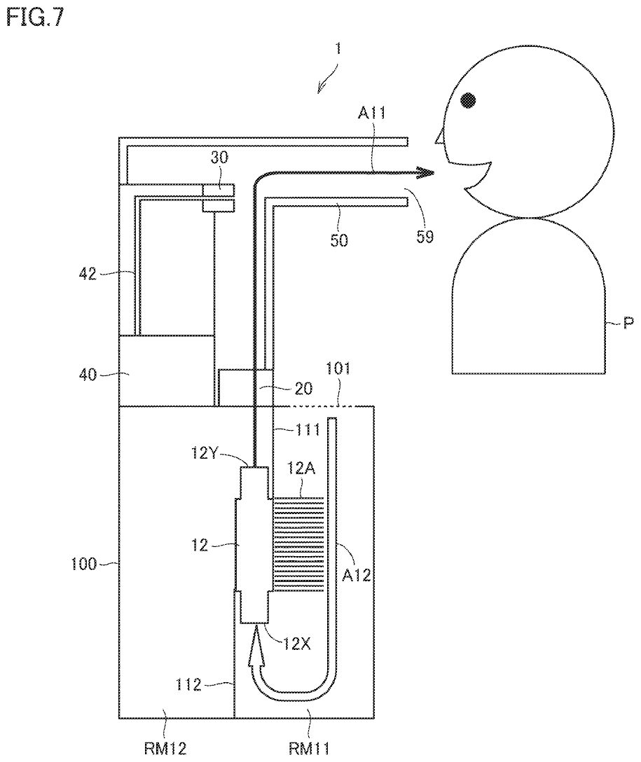

FIG. 7 is a schematic view illustrating a structure of a nebulizer of a seventh embodiment according to the present disclosure. The example illustrated in FIG. 7 is different from the example illustrated in FIG. 1 in that the nebulizer 1 further includes fins 12A attached to the pump 12. The fins 12A are an example of a heat sink for the pump 12. The fins 12A can promote the heat emission from the pump 12.

### Eighth Embodiment

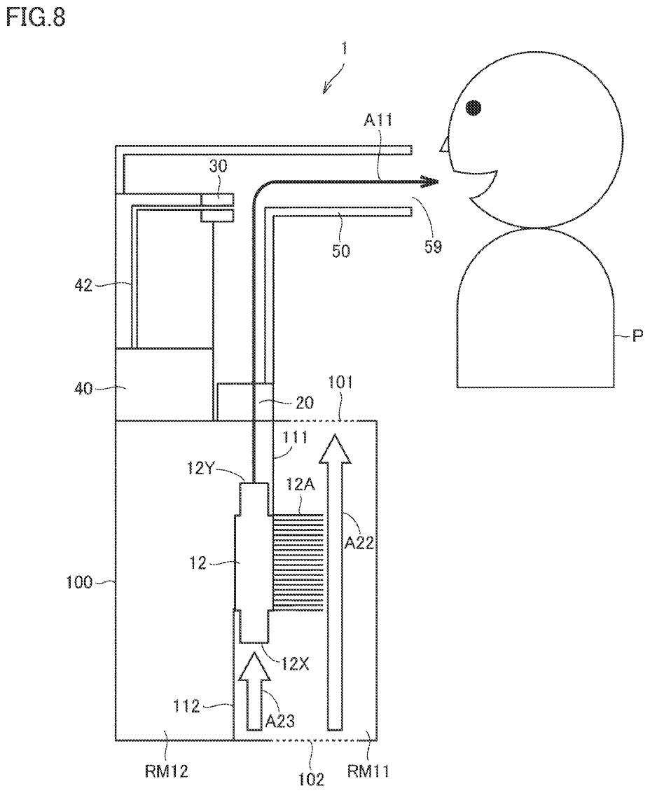

FIG. 8 is a schematic view illustrating a structure of a nebulizer of an eighth embodiment of the present disclosure. The example illustrated in FIG. 8 is different from the example illustrated in FIG. 2 in that the nebulizer 1 further includes the fins 12A attached to the pump 12. The fins 12A is an example of the heat sink for the pump 12. The fins 12A can promote the heat emission from the pump 12.

### Ninth Embodiment

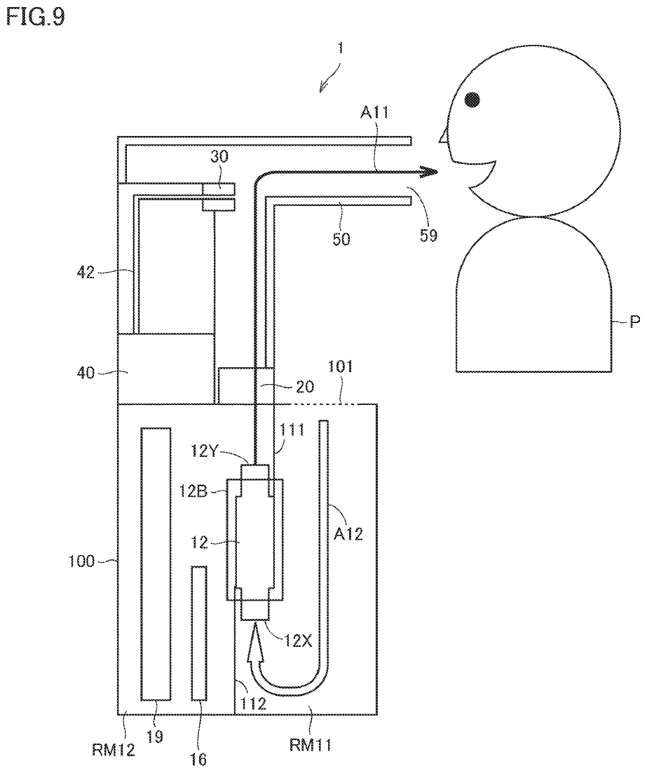

FIG. 9 is a schematic view illustrating a structure of a nebulizer of a ninth embodiment according to the present disclosure. In the example illustrated in FIG. 9, the housing 100 accommodates the board 16 and the battery 19. The nebulizer 1 further includes a pump cover 12B that covers the pump 12.

The pump cover 12B is an example of the heat sink for the pump 12. The pump cover 12B absorbs the heat produced by the pump 12 and prevents the heat from reaching the board 16 and/or the battery 19.

The air flow indicated by arrow A12 may introduce air into the pump cover 12B. In other words, the outer surface of the pump 12 covered by the pump cover 12B can be cooled by an air flow passing inside the pump cover 12B. In the example illustrated in FIG. 9, the outer surface of the pump 12 can be cooled by air flowing outside and inside the pump cover 12B. This further increases the cooling effect.

### Tenth Embodiment

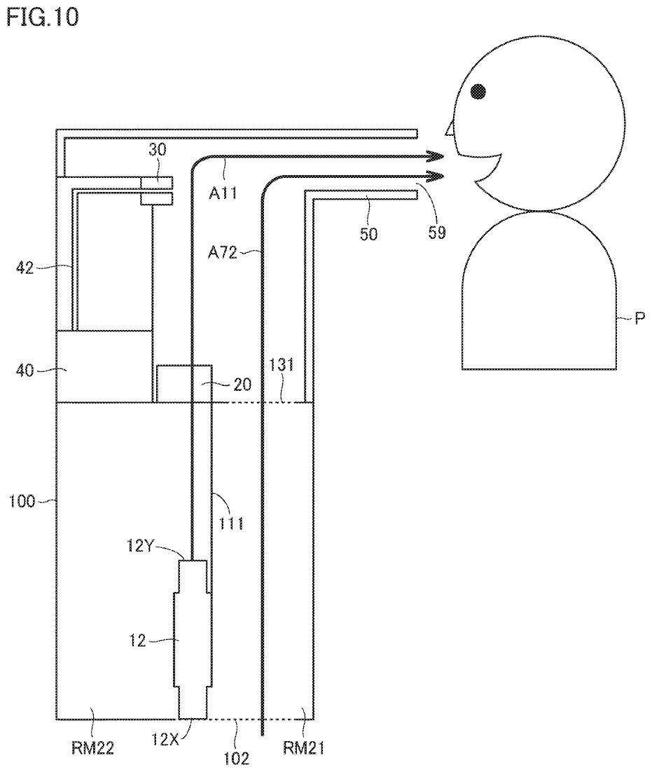

FIG. 10 is a schematic view illustrating a structure of a nebulizer of a tenth embodiment according to the present disclosure. In the example illustrated in FIG. 3, the inside (space RM21) of the housing 100 communicates with the outside of the housing 100 through the opening 101 formed at the top surface of the housing 100. On the other hand, in the example illustrated in FIG. 10, the inside of the housing 100 communicates with the inside of the mouthpiece 50 through an opening 131 formed at the top surface of the housing 100.

The air introduced into the housing 100 (i.e., into the space RM21) through the opening 102 is further introduced into the mouthpiece 50. Arrow A72 indicates an air flow of which the air is introduced in the space RM21 through the opening 102, passes through the mouthpiece 50, and is discharged out through the outlet 59.

The air nozzle 20 supplies compressed air into the mouthpiece 50, which produces negative pressure inside the mouthpiece 50. The negative pressure promotes the air flow indicated by arrow A72. At least the air flow indicated by arrow A72 can promote the heat emission from the pump 12.

### Eleventh Embodiment

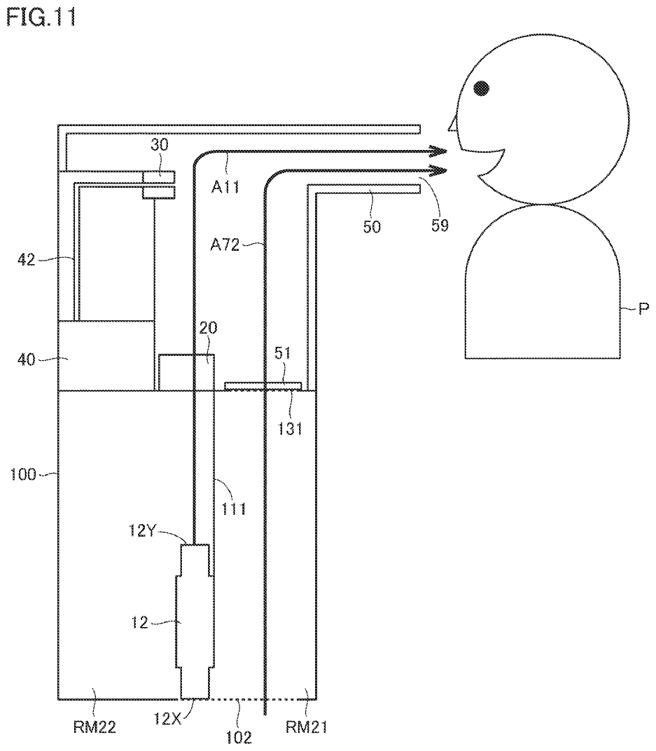

FIG. 11 is a schematic view illustrating a structure of a nebulizer of an eleventh embodiment according to the present disclosure. The example illustrated in FIG. 11 is different from the example illustrated in FIG. 10 in that the nebulizer 1 further includes a filter 51 that is disposed inside the mouthpiece 50 so as to cover an opening 131.

The mist of the liquid medicine is condensed into droplets inside the mouthpiece 50. The filter 51 prevents the droplets from flowing out through the opening 131. In other words, the filter 51 prevents the liquid medicine supplied by the atomizing nozzle 30 from flowing into the housing 100 through the opening 131 and also from flowing out of the housing 100 through the opening 102. The top surface of the filter 51 may be treated to be water-repellent and/or be structured to cause the liquid medicine to flow downward in order to prevent the liquid medicine from staying on the top surface. The filter 51 configured as above reduces the occurrence of hampering the air flow indicated by arrow A72. This improves the cooling effect of the housing 100 and promotes the discharge of the mist (aerosol) out of the mouthpiece 50.

### Twelfth Embodiment

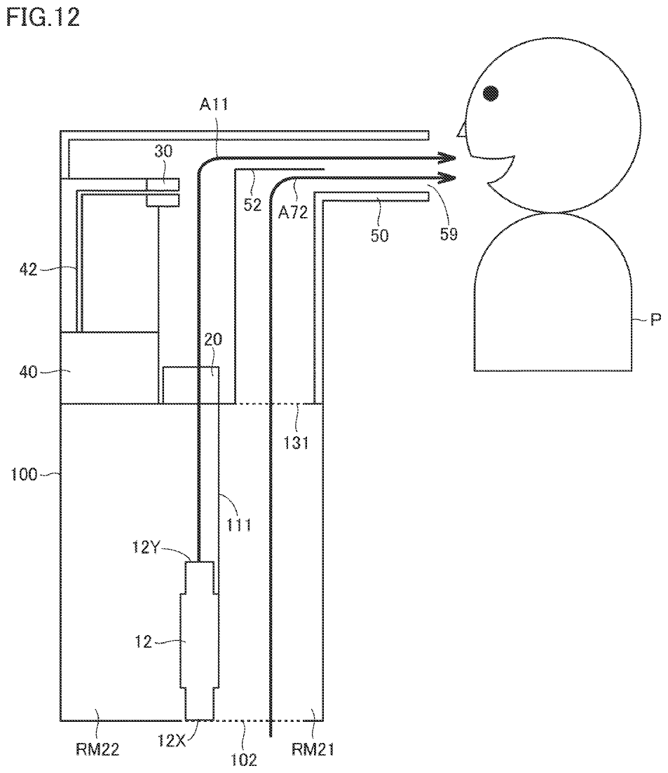

FIG. 12 is a schematic view illustrating a structure of a nebulizer of a twelfth embodiment according to the present disclosure. As illustrated in FIG. 12, the nebulizer 1 includes a guide plate 52 inside the mouthpiece 50. The guide plate 52 is provided to guide the air from the opening 131 toward the outlet 59.

The guide plate 52 is provided to prevent the liquid medicine condensed inside the mouthpiece 50 from flowing out through the opening 131. In other words, the guide plate 52 prevents the liquid medicine supplied by the atomizing nozzle 30 from flowing into the housing 100 through the opening 131 and also from flowing out of the housing 100 through the opening 102. In this regard, the guide plate 52 is an example of a screen member that screens the opening 131 at least partially from the atomizing nozzle 30 in the mouthpiece 50.

### Thirteenth Embodiment

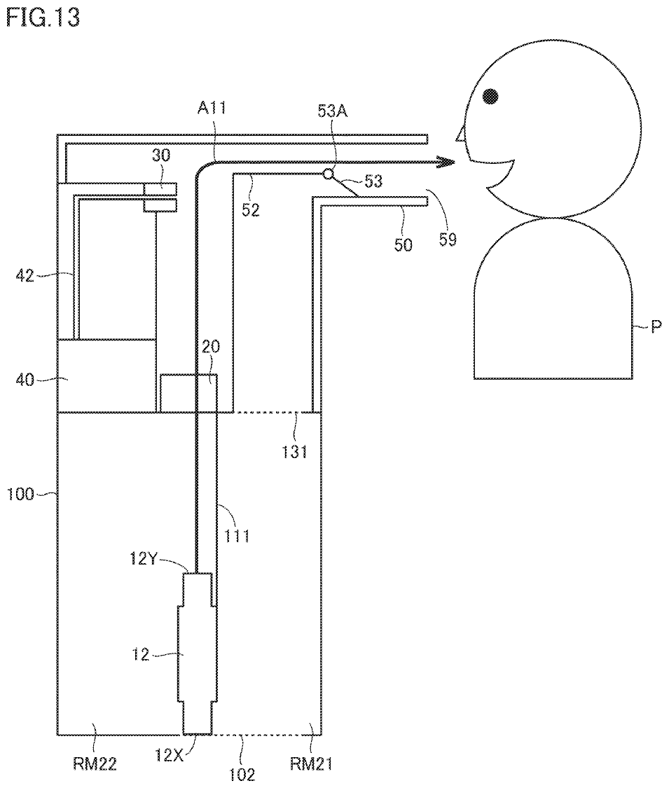

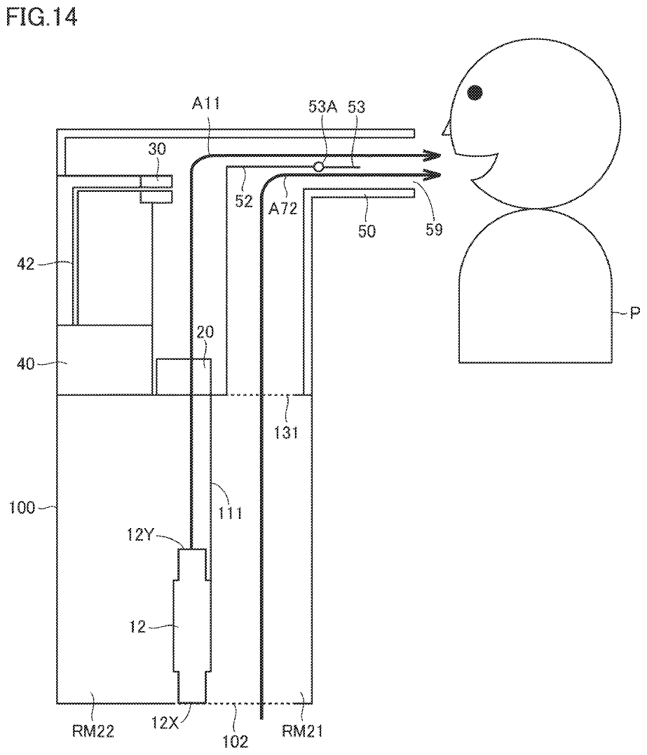

FIG. 13 is a schematic view illustrating a structure of a nebulizer of a thirteenth embodiment according to the present disclosure. The example illustrated in FIG. 13 is different from the example illustrated in FIG. 12 in that the nebulizer 1 further includes a valve 53 attached to the guide plate 52. A pivot 53A is attached to the guide plate 52. The valve 53 rotates about the pivot 53A. FIG. 14 is a view illustrating the nebulizer of the thirteenth embodiment in a state in which the valve 53 is rotated from the state illustrated in FIG. 13.

In FIG. 13, the valve 53 stays at the home position. For example, when a patient P inhales with the outlet 59 being put in the mouth and thereby generates a negative pressure downstream of the valve 53 and/or when air is sent into the opening 102 or the opening 131 to generate a positive pressure upstream of the valve 53, the valve 53 rotates about the pivot 53A and assumes a position as illustrated in FIG. 14. The valve 53 opens the channel for the air flow indicated by arrow A72 in the state of FIG. 14, and the valve 53 closes the channel for the air flow in the state of FIG. 13.

In the mouthpiece 50, the valve 53 screens the region where the opening 131 is present from the region where the atomizing nozzle 30 is present in the state of FIG. 13. On the other hand, in the state of FIG. 14, the valve 53 does not screen the region in which the opening 131 is present.

In the state of FIG. 13, closing the valve 53 can prevent the liquid medicine condensed inside the mouthpiece 50 from flowing out through the openings 131 and 102. In the state of FIG. 14, the air flow indicated by arrow A72 promotes the heat emission from the pump 12.

### Modifications, Etc

In the present specification, it has been described that various types of opening, such as the opening 101, are formed at the top surface or at the bottom surface of the housing 100. The positions of the openings, however, are not limited to the above, in other words, at the top surface and/or the bottom surface of the housing 100, insofar as the functions described herein can be implemented. The openings can be formed at the side surfaces of the housing 100.

The pumps 12 and 14 can be of any type, in other words, can be motor pumps or piezoelectric pumps.

The embodiments disclosed herein are to be construed, in all respects, not as limiting but as examples. It is intended that the scope of the present disclosure be set forth not in the above descriptions but in the claims in which all the modifications and alterations within the scope of the claims as well as the equivalents thereof are included.
1 nebulizer
12, 14 pump
12A fins
12B pump cover
12X, 14X inlet
12Y, 14Y, 59 outlet
16, 18 board
19 battery
20 air nozzle
30 atomizing nozzle
40 liquid medicine tank
42 piping
50 mouthpiece
51 filter
52 guide plate
53 valve
53A pivot
100 housing
100X inner surface
101, 102, 121, 122, 131 opening
111, 112 partition plate
RM11, RM12, RM21, RM22, RM31, RM32, RM33 space

1. A nebulizer comprising:
a pump for spraying a liquid reagent; and
a housing surrounding the pump, wherein
the housing having a space defined at least partially by an outer surface of the pump and by an inner surface of the housing and/or an element inside the housing,
at least one opening is provided to enable the space to communicate with an outside of the housing.

2. The nebulizer according to claim 1, wherein
the element inside the housing is a battery for driving the pump and/or a board having a control circuit for controlling the pump.

3. The nebulizer according to claim 1, wherein
the pump is disposed such that an inlet of the pump is exposed to the space.

4. The nebulizer according to claim 1, wherein
the at least one opening includes a first opening and a second opening.

5. The nebulizer according to claim 1, wherein
the pump is disposed such that an inlet of the pump is positioned at the at least one opening.

6. The nebulizer according to claim 1, further comprising:
a cover connected to the housing; and
a nozzle configured to supply the liquid reagent to an inside of the cover, wherein
air sent by the pump discharges the liquid reagent supplied from the nozzle to an outside of the cover, and
the space communicates with the inside of the cover through the at least one opening.

7. The nebulizer according to claim 6, further comprising:
a filter disposed at the at least one opening through which the space communicates with the inside of the cover.

8. The nebulizer according to claim 6, further comprising:
a screen member disposed inside the cover and screens a region inside the cover at least partially from the nozzle, the region communicating with the space.

9. The nebulizer according to claim 8, wherein
the cover has an outlet through which the liquid reagent is discharged out of the nebulizer,
the screen member serves as a part of a channel inside the cover, the channel guiding air introduced from the space toward the outlet, and
the cover further includes a valve to open/close the channel.

10. The nebulizer according to claim 1, further comprising:
a heat sink attached to the pump or disposed near the pump.

11. The nebulizer according to claim 2, wherein
the pump is disposed such that an inlet of the pump is exposed to the space.

12. The nebulizer according to claim 2, wherein
the at least one opening includes a first opening and a second opening.

13. The nebulizer according to claim 3, wherein
the at least one opening includes a first opening and a second opening.

14. The nebulizer according to claim 2, wherein
the pump is disposed such that an inlet of the pump is positioned at the at least one opening.

15. The nebulizer according to claim 2, further comprising:
a cover connected to the housing; and
a nozzle configured to supply the liquid reagent to an inside of the cover, wherein
air sent by the pump discharges the liquid reagent supplied from the nozzle to an outside of the cover, and
the space communicates with the inside of the cover through the at least one opening.

16. The nebulizer according to claim 3, further comprising:
a cover connected to the housing; and
a nozzle configured to supply the liquid reagent to an inside of the cover, wherein
air sent by the pump discharges the liquid reagent supplied from the nozzle to an outside of the cover, and
the space communicates with the inside of the cover through the at least one opening.

17. The nebulizer according to claim 4, further comprising:
a cover connected to the housing; and
a nozzle configured to supply the liquid reagent to an inside of the cover, wherein
air sent by the pump discharges the liquid reagent supplied from the nozzle to an outside of the cover, and
the space communicates with the inside of the cover through the at least one opening.

18. The nebulizer according to claim 5, further comprising:
a cover connected to the housing; and
a nozzle configured to supply the liquid reagent to an inside of the cover, wherein
air sent by the pump discharges the liquid reagent supplied from the nozzle to an outside of the cover, and
the space communicates with the inside of the cover through the at least one opening.

19. The nebulizer according to claim 2, further comprising:
a heat sink attached to the pump or disposed near the pump.

20. The nebulizer according to claim 3, further comprising:
a heat sink attached to the pump or disposed near the pump.
